(19) United States
(12) Patent Application Publication
(10) Pub. No.: US 2010/0198088 A1
(43) Pub. Date: Aug. 5, 2010

(54) METHOD, APPARATUS AND SYSTEM FOR DETECTION OF ARTERIAL STIFFNESS AND ARTERY TONUS BY PULSE CURVE GEOMETRY ANALYSIS

(76) Inventors: Michael Ortenberg, Kfar Yona (IL); Ronen Arbel, Tel Aviv (IL); Yoram Tal, Tel Aviv (IL)

Correspondence Address: Pearl Cohen Zedek Latzer, LLP, 1500 Broadway, 12th Floor, New York, NY, 10036, US

(21) Appl. No.: 12/598,181

(22) PCT Filed: May 1, 2008
(86) PCT No.: PCT/IL08/00595

§ 371 Date: Feb. 15, 2010

Related U.S. Application Data
(60) Provisional application No. 60/924,129, filed on May 1, 2007.

(51) Int. Cl. A61B 5/0255

(52) U.S. Cl. 600/501

(57) ABSTRACT

System and method for determining a condition of a cardiovascular system are provided. One or more pulse curves may be obtained. At least two points on the pulse curve, substantially including a dicrotic notch may be selected. A first parameter pertaining to a section of the pulse curve contained by the selected points may be calculated. A second parameter pertaining to a curve other than the pulse curve and traversing the selected points may be calculated. A notch coefficient may be computed by relating the first parameter to the second parameter. Other embodiments are described and claimed.

### BACKGROUND

Arterial stiffness increases both with age and in certain disease states associated with increased cardiovascular risk, including hypertension, diabetes mellitus, hypercholesterolemia and end-stage renal failure. Various methods and/or techniques for analyzing and/or evaluating a condition of a cardiovascular system exist. There is a need in the art for additional indicators that may be used to better determine a condition of a cardiovascular system.

There is a need for a method and apparatus to perform an evaluation of endothelial functions in the coronary arteries. Such evaluation may enable predicting future undesirable cardiovascular events by detecting abnormalities associated with a functionality of the cardiovascular system.

### BRIEF DESCRIPTION OF THE DRAWINGS

Embodiments of the invention are illustrated by way of example and not limitation in the figures of the accompanying drawings, in which like reference numerals indicate corresponding, analogous or similar elements, and in which:

FIG. 1 shows an exemplary pulse curve helpful in understanding according to embodiments of the invention;

FIG. 2 shows an exemplary pulse curve helpful in understanding embodiments of the invention;

FIG. 3 shows an exemplary pulse curve and a fourth derivative curve according to embodiments of the invention;

FIG. 4 shows a histogram of field test results demonstrating embodiments of the invention;

FIG. 5 shows an exemplary system according to embodiments of the invention; and

FIG. 6 shows an exemplary flowchart according to embodiments of the invention.

### DETAILED DESCRIPTION OF EMBODIMENTS OF THE INVENTION

In the following detailed description, numerous specific details are set forth in order to provide a thorough understanding of the invention. However, it will be understood by those of ordinary skill in the art that the invention may be practiced without these specific details. In other instances, well-known methods, procedures, components, modules, units and/or circuits have not been described in detail so as not to obscure the invention.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing devices, that may manipulate and/or transform data represented as physical (e.g., electronic) quantities within the computer's registers and/or memories into other data similarly represented as physical quantities within the computer's registers and/or memories or other information storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. For example, “a plurality of parameters” may include two or more parameters.

Unless explicitly stated, the method embodiments described herein are not constrained to a particular order or sequence. Additionally, some of the described method embodiments or elements thereof can occur or be performed at the same point in time.

It will be recognized that according to embodiments of the invention, any volumetric measurement pertaining to a cardiovascular system and further capable of producing information that may be used for analyzing a pulse curve as described below may be used. For example, an invasive sensor inserted into a blood vessel may be used. Another example may be where obtaining a pulse curve comprises utilizing a photoplethysmograph (PTG) measuring device. For the sake of simplicity and clarity, in the specification below, PTG pulse curve is referred to. It will be recognized that referring to PTG pulse curve is done for simplicity and a pulse curve obtained by any other applicable means may be used without departing from the scope of the invention.

According to embodiments of the invention, in order to estimate a condition of a cardiovascular system, e.g., an artery stiffness parameter, the geometry of a photoplethysmograph (PTG) pulse curve may be analyzed. Geometry of a PTG pulse curve may be directly linked to the blood flow in the arteries and may correlate well with a measure of stiffness of the large arteries. Such geometry may additionally correspond to changes in the pulse wave velocity (PWV) of pressure waves in the aorta and large arteries.

According to embodiments of the invention, an analysis of at least two regions of a PTG pulse curve may be performed. Reference is made to FIG. 1 showing an exemplary PTG pulse curve 101 and two associated regions 110 and 120. Region 110 may be characterized by an augmentation index (AI) that may predominantly reflect a stiffness condition associated with changes pertaining to an age of the subject. As shown in FIG. 1, region 120 includes a dicrotic notch 130 and a dicrotic wave 135. According to embodiments of the invention, the duration, relative location and minimal amplitude of a dicrotic notch and/or a dicrotic wave may be calculated and may further be used to compute indicators reflecting a condition of the cardiovascular system.

According to embodiments of the invention, an artery condition may be characterized by a notch coefficient (NC) parameter that may be associated with a dicrotic notch and/or a dicrotic wave such as dicrotic notch 130 and a dicrotic wave 135 shown within region 120. According to embodiments of the invention, such condition may be a endothelial function condition and/or an arterial stiffness condition.

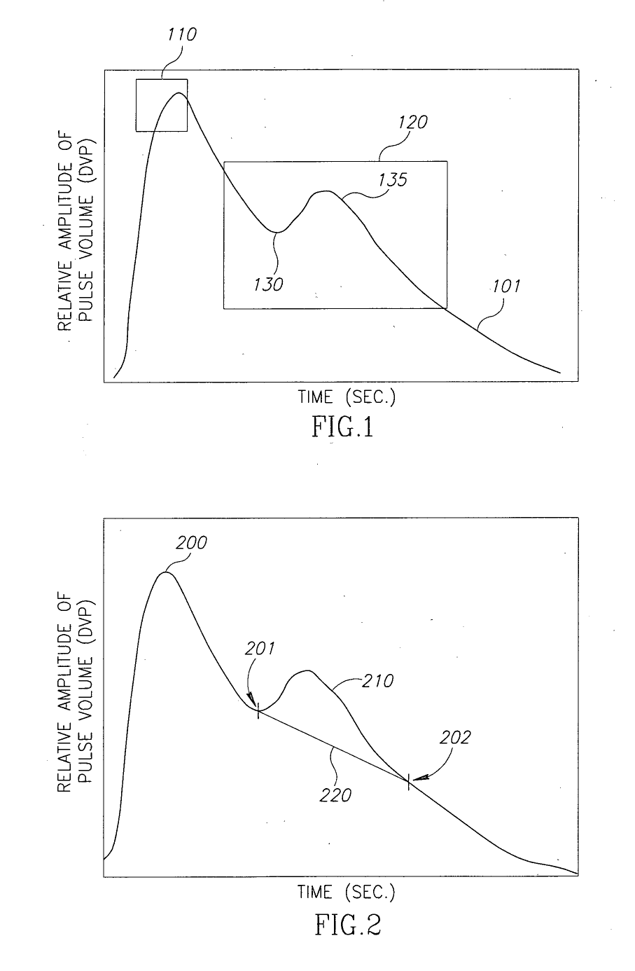

Reference is made to FIG. 2 showing an exemplary PTG pulse curve 200. PTG pulse curve 200 may include a section denoted by 210 where section 210 may be delimited by points 201 and 202. According to embodiments of the invention, points 201 and 202 may mark a start and end point respectively of a dicrotic wave and accordingly, section 210 may correspond with such dicrotic wave. According to embodiments of the invention, a straight line as shown by 220 may connect points 201 and 202.

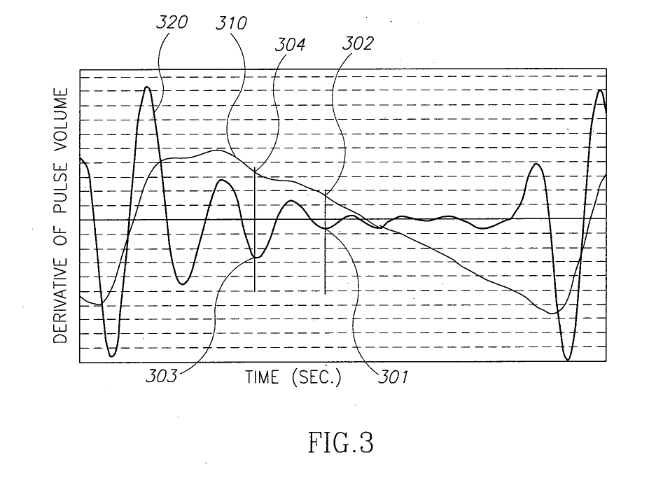

Reference is now made to FIG. 3 showing an exemplary PTG pulse curve and a graphic representation of its fourth derivative with respect to time (δPTG⁴/δt⁴), both helpful in understanding embodiments of the invention. The exemplary PTG pulse curve, denoted 310 and the fourth derivative curve of the PTG pulse curve, denoted 320 may be used to locate start and end or edge points of a dicrotic wave. According to embodiments of the invention, the minimum values of the fourth derivative curve 320, denoted by points 301 and 303 may be used to locate edge points of a dicrotic wave of the PTG pulse curve. According to embodiments of the invention, a first edge point 302 and a second edge point 304 may be located by an intersection of respective vertical lines connecting the minimum values 301 and 303 with the associated PTG pulse curve 310 as shown by FIG. 3.

Referring back to FIG. 2, according to embodiments of the invention, a notch coefficient (NC) parameter may be defined by relating one or more parameters associated with section 210 with one or more parameters associated with line 220. For example, a NC parameter may be defined as the arc, curve or section length of the dicrotic wave as shown by 210 divided by the length of the straight line connecting the dicrotic wave's edge points as shown by line 220. According to embodiments of the invention, with reference to FIG. 2, a NC parameter or indicator may be defined by (L1/L2) where L1 is the length of the dicrotic wave section, e.g., section 201 and L2 is the length of a straight line connecting the edge points of a dicrotic wave, e.g., line 220.

Computing a NC parameter as described above may yield substantially indiscrete values, typically between 1.3 and 1.5. According to embodiments of the invention, a NC parameter may be expressed as follows:

NC=e^{(L1/L2)}−e,

wherein “e” is the base of the natural logarithm, known as Euler's number.

Expressed as shown above, the range of such NC parameter values may be expanded, for most cases, to a range between 0 and about 1.5.

According to embodiments of the invention, a notch coefficient (NC) parameter may be calculated by relating any combination of parameters associated with a section such as section 210 and a line such as line 220 to any other such combination. For example, a NC parameter may be computed as follows:

NC=(αL1+βL2)/(γL1+δL2)

where L1 is the length of section 210 and L2 is the length of line 220 and where α, β, γ and δ may be any coefficients including zero (“0”) or a fraction such as ⅓.

For example, when α equals 0.5, β equals 0.5, γ equals 0 and δ equals 2, the NC parameter may be computed as NC=(0.5*L1+0.5*L2)/2*L2. According to embodiments of the invention, the coefficients α, β, γ and δ may be altered in order to fine tune or otherwise adjust the calculation of the NC parameter. For example to better correspond with various aspects such as age or weight of a tested subject. For example, a predefined set of values assigned to α, β, γ and δ may be used for young populations while another set of values assigned to α, β, γ and δ may be used for elderly subjects. As described above, a NC parameter may accordingly be expressed as:

NC=e^{(αL1+βL2)/(γL1+δL2)}−e,

where “e” is defined as described above.

According to embodiments of the invention, a NC parameter may be used as an indicator of arterial stiffness and/or artery tonus in a cardiovascular system. In order to evaluate the NC parameter, a field test was conducted, where a NC according to embodiments of the invention, was computed for 280 subjects with no known cardiovascular disease and 124 subjects suffering from known heart diseases or cardiovascular disease (CVD).

The field test included obtaining a PTG waveform from all 280 subjects. The PTG waveform was obtained by a Photoplethysmograph (PTG) sensor placed on a finger tip of the tested subject. An analog-to-digital (A/D) converter was used to digitize the signals received from the PTG sensor. Digital signals produced by the A/D converter were provided as input to a computer. It will be recognized that any applicable method for obtaining a PTG from a subject may be used without departing from the scope of the invention. The obtained PTG waveforms were used to compute a NC parameter value for each of the tested subjects.

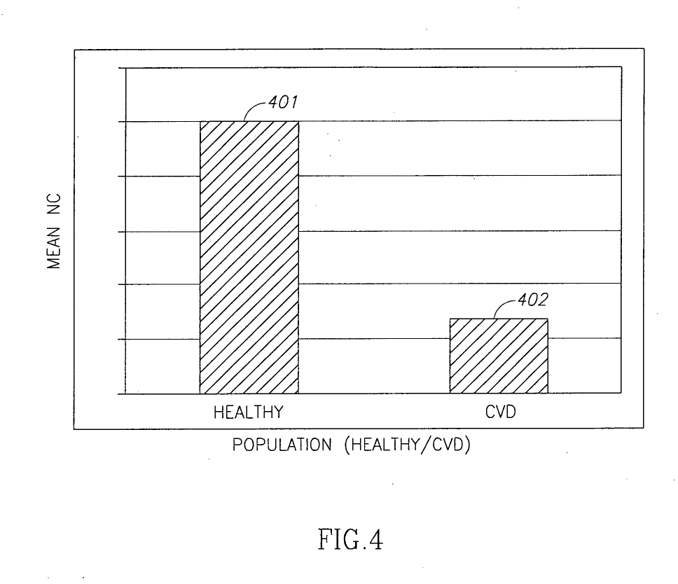

Reference is now made to FIG. 4 showing a column histogram representation of results obtained by the field test. Column 401 represents NC values associated with the healthy population and column 402 represents NC values associate with the unhealthy population. As shown by FIG. 4, higher NC parameter values, were obtained for healthy subjects, relative to lower NC values that were obtained from the unhealthy subjects. The results obtained as shown by FIG. 4 may indicate a high relevancy of the NC parameter to the condition of the tested cardiovascular system. Accordingly, a NC parameter may be used as an indicator of a condition of a cardiovascular system, in particularly, a stiffness condition of the aortic vessels.

According to embodiments of the invention, a test result may be computed by comparing or otherwise relating a NC parameter calculated as described above to a predefined threshold or value or a set of predefined thresholds. For example, a low threshold may be defined such that NC parameter values below such threshold are considered to indicate a risk, for example a possible of CVD. Alternatively, a range of values may be defined and a good or acceptable result may be one contained by such defined range. According to embodiments of the invention, predefined thresholds may be defined for various categories of subjects. For example, a first set of thresholds may be used for males while a different may be used for females, alternatively, such thresholds sets may be according to age, general fitness or any other applicable parameter, condition or circumstances.

According to embodiments of the invention, an apparatus may be used for obtaining a pulse curve such as a PTG pulse curve from a cardiovascular system. For example, an apparatus comprising computing means linked to suitable PTG measuring means. Such apparatus, or another apparatus, possibly operatively connected to the measuring apparatus may further compute parameters or indicators such as a NC parameter. Such parameters and/or indicators may be used in order to assess a condition of a cardiovascular system, in particular arterial stiffness and artery tonus conditions.

According to embodiments of the invention, the measuring, computing or another device may further present graphically, numerically or in any other applicable way computed results, parameters and/or indicators. For example, a computing device comprising a display may present indicators or parameters such as a NC numerically or otherwise. Such device may further compute and/or display a comparison of one or more parameters or indicators with a reference set of relevant parameters or indicators. For example, a NC parameter computed for a specific patient may be visually compared to a reference NC parameter of a reference, possibly average, human. For example, a reference human of same age, gender etc.

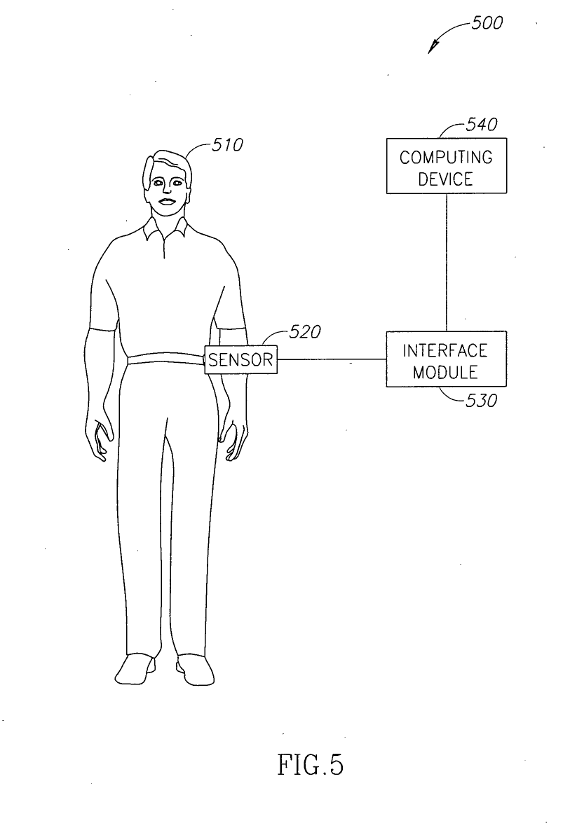

Reference is now made to FIG. 5 showing an exemplary system used to obtain a PTG waveform from a subject according to embodiments of the invention. A system 500 may compute, calculate or otherwise derive indicators and/or parameters such as a notch coefficient. System 500 may include a sensor 520 to collect data from the subject, an interface module 530 coupled to sensor 520 and a computing device 540 coupled to interface module 530. According to embodiments of the invention and as shown by 520, a sensor or other information or data collecting device may be used to collect data from subject 510. Such sensor may be any suitable sensing device and applicable circuitry. For example, sensor 520 may be an off the shelf sensing device such as a saturation of oxygen in arterial blood flow (SaO₂) sensing device or any photoplethysmograph sensing device.

According to embodiments of the invention and as shown by 530, sensor 520 may be operatively connected to interface module 530. According to embodiments of the invention, interface module 530 may perform various tasks. For example, module 530 may perform a conversion of analog information to digital information (A/D) or it may perform up-sampling or down-sampling of data received from sensor 520, interface module may also translate or transform commands sent from computing device 540 to sensor 520 as well as provide electric power to sensor 520. According to embodiments of the invention, interface module 530 may be operatively connected to computing device 540.

Some embodiments of the present invention may be implemented in software for execution by a processor-based system such as computing device 540, as shown in FIG. 5. For example, embodiments of the invention may be implemented in code and may be stored on a storage medium having stored thereon instructions which can be used to program a system to perform the instructions. The storage medium may include, but is not limited to, any type of disk including floppy disks, optical disks, compact disk read-only memories (CD-ROMs), rewritable compact disk (CD-RW), and magneto-optical disks, semiconductor devices such as read-only memories (ROMs), random access memories (RAMs), such as a dynamic RAM (DRAM), erasable programmable read-only memories (EPROMs), flash memories, electrically erasable programmable read-only memories (EEPROMs), magnetic or optical cards, or any type of media suitable for storing electronic instructions, including programmable storage devices.

According to embodiments of the invention, computing device 540 may be any suitable computing device. For example, computing device 540 may be a personal computer, a desktop computer, a mobile computer, a laptop computer, a set-top box, a notebook computer, a workstation, a server computer, a tablet computer, a network appliance, personal digital assistant (PDA). Computing device 540 may further be equipped and configured to communicate with one or more computing devices over a communication network. According to embodiments of the invention, sensor 520 may obtain a PTG waveform data from subject 510. Sensor 520 may communicate such obtained waveform data to computing device 540 via interface module 530. According to embodiments of the invention, computing device 540 may use such data to calculate, compute or otherwise derive a notch coefficient pertaining to the cardiovascular system of subject 510.

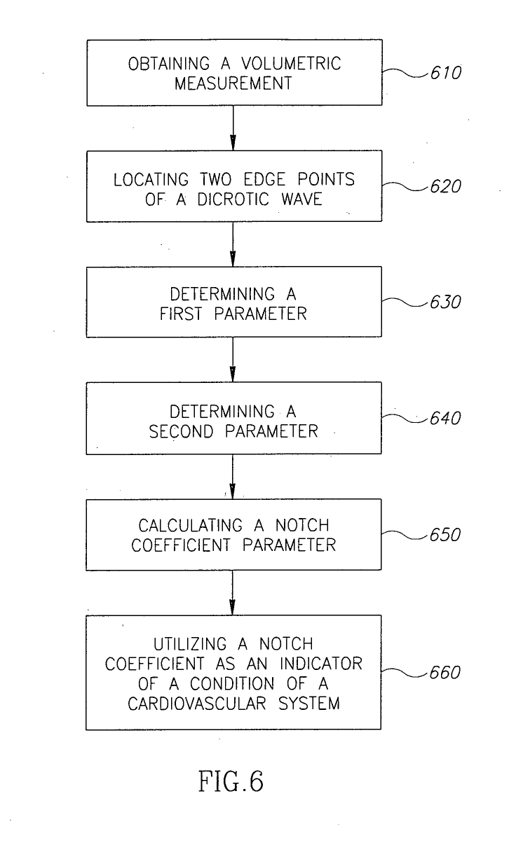

Reference is now made to FIG. 6 showing an exemplary flowchart that may, according to embodiments of the invention, be used to derive indicators and/or parameters such as a notch coefficient. According to embodiments of the invention and as shown by 610, the flow may include obtaining at least one volumetric pulse curve measurement. For example, sensor 520 may obtain such pulse curve using a photoplethysmograph from subject 510 and may further communicate such data to computing device 540 as described above. According to embodiments of the invention, obtaining a volumetric pulse curve may be performed by any suitable means. For example, an invasive sensor or any other suitable means may be used.

According to embodiments of the invention and as shown by block 620, the flow may include locating two edge points of a dicrotic wave. For example, computing device 540 may perform an analysis of an obtained PTG pulse curve and locate two edge points of an associated dicrotic wave as described above. According to embodiments of the invention and as shown by block 630, the flow may include calculating a first parameter. For example, such first parameter may be the length of a section of the obtained PTG pulse curve where the section is delimited by the edge points calculated as described above. According to embodiments of the invention and as shown by block 640, the flow may include calculating a second parameter. For example, such second parameter may be the length of a line connecting the edge points calculated as described above. For example, such line may be the shortest possible line connecting the two edge points, namely, a straight line.

According to embodiments of the invention and as shown by block 650, the flow may include calculating a notch coefficient parameter. For example, a notch coefficient parameter may be calculated by dividing the length of the PTG curve pulse section described above by the length of the line described above. According to embodiments of the invention, any suitable equation, method or procedure utilizing such parameters may be used to compute, calculate or otherwise derive a notch coefficient as shown by block 650. According to embodiments of the invention, a notch coefficient may further be expressed as an exponent of a ratio of the lengths described above or as an exponent of any number or metric derived by a mathematical operation involving lengths or other parameters pertaining to the section of the PTG curve and the line described above.

According to embodiments of the invention and as shown by block 660, the flow may include calculating an indicator of a cardiovascular condition. According to embodiments of the invention, a notch coefficient may be used to calculate an indicator of a cardiovascular condition. For example, a value of a notch coefficient may be compared or otherwise related to a predefined value and an indicator may be computed according to relational aspects. For example, the difference or distance of a notch coefficient value from a predefined value may be used as an indicator.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

What is claimed is:

1. A method for detecting a condition of a cardiovascular system comprising:
obtaining a pulse curve related to said cardiovascular system;
locating two edge points of a dicrotic wave associated with said pulse curve;
determining a first parameter pertaining to a section of said pulse curve wherein said section is delimited by said two edge points;
determining a second parameter pertaining to a line connecting said two edge points;
calculating a notch coefficient parameter by using said first parameter and said second parameter; and
utilizing said notch coefficient parameter as an indicator of said condition.

2. The method of claim 1, wherein said line is a straight line.

3. The method of claim 1, wherein said first parameter is a length of said section of said pulse curve and wherein said second parameter is a length of said line.

4. The method of claim 1, wherein calculating said notch coefficient parameter comprises dividing said first parameter by said second parameter.

5. The method of claim 1, wherein utilizing said notch coefficient parameter comprises determining the condition of the cardiovascular system by comparing said notch coefficient parameter to a predefined threshold.

6. The method of claim 1, wherein said condition is an arterial stiffness.

7. The method of claim 1, wherein said condition is an artery tonus condition.

8. The method of claim 1, calculating the notch coefficient parameter comprises calculating e^{(L1/L2)}−e, wherein L1 is a length of said section of said pulse curve and wherein L2 is a length of said line.

9. The method of claim 1, wherein obtaining the pulse curve comprises utilizing a photoplethysmograph (PTG) measuring device.

10. An article comprising a computer-storage medium having stored thereon instructions that, when executed by a processing platform, result in:
obtaining a pulse curve related to a cardiovascular system;
locating two edge points of a dicrotic wave associated with said pulse curve;
determining a first parameter pertaining to a section of said pulse curve wherein said section is delimited by said two edge points;
determining a second parameter pertaining to a line connecting said two edge points;
calculating a notch coefficient parameter by using said first parameter and said second parameter; and
utilizing said notch coefficient parameter to compute an indicator of a condition of said cardiovascular system.

11. The article of claim 10, wherein the instructions when executed to calculate the notch coefficient further result in:
calculating said notch coefficient parameter by dividing a length of said section of said pulse curve by a length of said line.

12. The article of claim 10, wherein the instructions when executed further result in comparing said notch coefficient parameter to a predefined threshold; and determining the condition of the cardiovascular system.

13. A system comprising:
a sensor to obtain a volumetric measurement related to a cardiovascular system of a subject;
an interface module to convert information received from said sensor;
a computing device to receive the information from said interface module and to calculate a notch coefficient based on said information by:
obtaining a pulse curve related to the cardiovascular system;
locating two edge points of a dicrotic wave associated with said pulse curve;
determining a first parameter pertaining to a section of said pulse curve wherein said section is delimited by said two edge points;
determining a second parameter pertaining to a line connecting said two edge points;
calculating a notch coefficient parameter by using said first parameter and said second parameter; and
utilizing said notch coefficient parameter as an indicator of said condition.

14. The system of claim 13, wherein said computing device to calculate the notch coefficient parameter by dividing a length of said section of said pulse curve by a length of said line.

15. The system of claim 13, wherein said computing device is further to compare said notch coefficient parameter to a predefined threshold and to determine the condition of the cardiovascular system based on said comparing.

16. The system of claim 13, wherein said computing device is to calculate the notch coefficient parameter by calculating e^{(L1/L2)}−e, wherein L1 is a length of said section of said pulse curve and wherein L2 is a length of said line.
